(19) United States
(12) Patent Application Publication
(10) Pub. No.: US 2021/0275052 A1
(43) Pub. Date: Sep. 9, 2021

(54) METHODS, SYSTEMS, AND DEVICES FOR INCORPORATING VIBRATION THERAPY AND VIBRATION FEEDBACK INTO A BIOELECTRIC TEST PROBE

(71) Applicant: Vine Medical LLC, Saint George, UT (US)

(72) Inventor: Phillip W. Dietz, Saint George, UT (US)

(73) Assignee: Vine Medical LLC, Saint George, UT (US)

(21) Appl. No.: 17/163,959

(22) Filed: Feb. 1, 2021

Related U.S. Application Data
(60) Provisional application No. 62/968,077, filed on Jan. 30, 2020.

(51) Int. Cl. A61B 5/0532; A61H 23/02; A61B 5/00; A61H 39/02

(52) U.S. Cl. CPC A61B 5/0532; A61H 23/02; A61H 2201/50; A61B 5/7455; A61H 39/02; A61B 5/746

(57) ABSTRACT

Systems, methods, and devices for taking bioelectric measurements of a test subject using an automated bioelectric measurement system. A bioelectric testing device may include a conductive tip that is disposed at a distal end of the bioelectric testing device, and a vibration motor for vibrating the conductive tip during bioelectric testing of a test subject. The bioelectric testing device may further include a motor that applies a motor output force to the conductive tip during bioelectric testing to maintain proper force between a test subject and the conductive tip during the bioelectric testing. The one or more vibration motors may vibrate to provide alerts about operation or operability of the bioelectric testing device to a technician or test subject, to improve conductance between the device and the test subject, or to decrease discomfort and pain for the test subject.

### CROSS-REFERENCE TO RELATED APPLICATIONS

This application claims the benefit of U.S. Provisional Application No. 62/968,077, filed Jan. 30, 2020, titled, “METHODS, SYSTEMS, AND DEVICES FOR INCORPORATING VIBRATION THERAPY AND VIBRATION FEEDBACK INTO A BIOELECTRIC TEST PROBE,” which is incorporated herein by reference in its entirety, including but not limited to those portions that specifically appear hereinafter, the incorporation by reference being made with the following exception: In the event that any portion of the above-referenced provisional application is inconsistent with this application, this application supersedes the above-referenced provisional application.

### TECHNICAL FIELD

The disclosure is related to bioelectric test probes and more particularly, but not necessarily entirely, to incorporating vibration therapy and vibration feedback in a bioelectric test probe. The incorporation of the vibration therapy enables a test probe tip to: (1) penetrate the outer cornified layer of a test subject's skin and seat closer to meridian points with less applied pressure from the technician, (2) reduce patient discomfort, and (3) provide feedback to the technician and patient throughout the test procedure.

### BACKGROUND

The electrical conductance of body tissue can be measured and analyzed to gather information about a body's condition and to aid in diagnosing certain conditions. One form of measuring electrical conductance of body tissue is Electroacupuncture According to Voll (EAV). EAV and other electrical conductance diagnostic systems measure conductance levels at meridian points of the body. These electrical conductance diagnostic systems are used by some health practitioners to gain additional insight into the body's compatibility with certain supplements or materials, whether certain pathogens or toxins reside in the body, dental conditions in the body, and more.

Current devices for measuring skin conductivity include a bioelectric test probe that is used to measure conductivity of meridian points, acupressure points, and other tissues. In the probe, a grip area is located adjacent to a conductive tip, allowing the technician to firmly grip the device while taking measurements. The entire device is enclosed in a non-conductive housing to prevent contamination in the measurements. A cable connects the device to an EAV or other system to receive the measurement and calculate the skin conductivity. When taking measurements, a technician will position the probe over the tissue and apply the conductive tip to the surface of the skin to measure conductivity at a sample site.

Meridian points are located under the skin, and the skin can act as an insulator and increase the difficulty of taking accurate measurements. To compensate for this difficulty due to insulation in the skin, a texture may be added to the tip surface to help the tip penetrate through the cornified outer layer of the skin without puncturing the skin. In addition to adding a texture to the tip surface, a technician may increase the pressure with which the tip is applied to the tissue over the meridian point. Although the increased pressure may help penetrate through insulating skin layers and decrease the insulation effect of skin, the increased pressure may lead to discomfort and pain in many patients. In addition to the difficulty in penetrating the insulating outer layers of the skin, current bioelectric test probes do not incorporate systems to provide rapid feedback for the technician or patient throughout the test procedure.

In light of the foregoing, disclosed herein are systems, methods, and devices for incorporating vibration therapy and vibration feedback within a bioelectric test probe to better penetrate the insulating outer skin layer without puncturing the skin, reduce discomfort during the test sequence, and provide feedback to the technician and patient during the test procedure.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a perspective view of an embodiment of a bioelectric measurement system, including a bioelectric test device or probe, a reference module (e.g., grounding device or hand mass), and a bioelectric measurement analyzing device.

FIG. 1B is a perspective view of bioelectric test device or probe in contact with a test subject.

FIG. 2A illustrates a cutaway view of an embodiment of a bioelectric test device or probe, including a conductive tip, a linear bearing, a switch, a circuit board, a circuit board mounted vibration device, a shaft, a shaft mounted vibration device, a motor, a non-conductive probe body and a body mounted vibration device.

FIG. 2B illustrates a front view of a head of an embodiment of a bioelectric test device or probe including a primary conductive tip and ancillary conductive tips.

FIG. 2C illustrates a front view of a head of an embodiment of a bioelectric test device or probe including a primary conductive tip and an ancillary conductive tip.

FIG. 3 illustrates an embodiment of a method for coupling vibration therapy with output force in a bioelectric test device or probe to penetrate the cornified layer of skin more effectively and seat the conductive tip closer to the meridian point while reducing pain and discomfort for the patient.

FIG. 4 illustrates an embodiment of a method for incorporating vibration feedback in a bioelectric test device or probe to provide enhanced communication to the patient during the test procedure.

FIG. 5 illustrates an embodiment of a method for incorporating vibration feedback in a bioelectric test device or probe to provide enhanced communication to the technician performing the test procedure.

FIG. 6 is a block diagram illustrating an example computing device.

### DETAILED DESCRIPTION

Disclosed herein are systems, methods, and devices for incorporating vibration therapy and vibration feedback in a bioelectric test device or probe to increase the accuracy of bioelectric measurements and reduce patient discomfort and pain during testing procedures. The bioelectric test device or probe may be used in conjunction with an electrical conductance diagnostic system such as an Electroacupuncture According to Voll (EAV) or other electrodermal sensor systems.

In the following description, for purposes of explanation and not limitation, specific techniques and embodiments are set forth, such as particular techniques and configurations, in order to provide a thorough understanding of the system and device disclosed herein. While the techniques and embodiments will primarily be described in context with the accompanying drawings, those skilled in the art will further appreciate that the techniques and embodiments may also be practiced in other similar devices.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the systems, methods, and devices for measuring temperature in an automated bioelectric measurement system through a thermal sensor and using temperature readings to regulate fan activity, motor output force, and/or bioelectric measurement device operation are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of Whether or not those elements are shown or described in another embodiment or figure. In other words, elements in the figures may be interchangeable between various embodiments disclosed herein.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Electroacupuncture According to Voll (EAV) devices can be deployed to measure conductance levels at meridian points in a body. An EAV device is a sensitive ohm meter for measuring resistance in the body. The resistance of a material, tissue, meridian pathway, and so forth can be assessed to calculate the conductivity of the material, tissue, or meridian pathway. A material with a lower resistance measurement will have a higher conductivity.

To detect resistance, an ohm meter (such as an EAV device) applies a small direct current flow through a material. Resistance measures the relative difficulty for current to flow through the material. Electrical conductors allow current to flow easily and have a correspondingly low resistance. Electrical insulators restrict current flow through and have a correspondingly high resistance. Ohm's Law applies to materials with a proportional relationship between voltage, current, and resistance according to:

V=RI

where V is voltage (measured in volts), I is current (measured in amps) and R is resistance (measured in ohms). Conductivity is the reciprocal of resistivity, expressed mathematically as 1/R and indicates a degree to which a specified material conducts electricity.

Human tissue generally has a resistance of about 98,000 Ohms between the tissue and ground. Meridian points have a general resistance of about 5,000 Ohms between the meridian point and ground. This means that meridian points throughout the human body are about twenty times more conductive than the tissue surrounding the points. This large differential in conductivity makes it possible to locate meridian points and to be very consistent in verifying the points with an EAV device.

An embodiment of the disclosure is a system for sensing the electrical conductance of a material such as body tissue. The system may sense the bio-conductivity of body tissue such as skin or some other tissue. An embodiment of the system includes an electrodermal sensor for contacting a test subject's skin and reading the electrical conductance of the test subject's skin. The electrodermal sensor may include one or more probe tips positioned on the electrodermal sensor to contact a site of the test subject's skin. In an embodiment, each of the one or more probe tips is independent and takes independent measurements of the test subject's skin.

The measurements taken by the system can be assessed for determining a skin resistance measurement and/or a meridian conductivity measurement for the test subject. The meridian conductivity measurement may include a meridian stress assessment for measuring energy associated with acupuncture meridians. The measurements can be used in multiple healthcare practices such as bio resonance therapy, bio-energy regulatory techniques, biocybernetics medicine, computerized electrodermal screening, computerized electrodermal stress analysis, electrodermal testing, limbic stress assessment, meridian energy analysis, point testing, and others.

However, the measurements taken by an electrodermal sensor can be inaccurate and ineffective if the test subject has nontypical skin conductivity. Many of the treatments and diagnoses determined based on electrodermal sensor readings are based on typical skin conductivity and cannot be effectively applied to test subjects with nontypical skin conductivity or if accurate readings cannot be consistently recorded. The outer cornified layer of skin can act as an insulator, increasing the difficulty of obtaining consistent and accurate skin conductivity readings with an electrodermal sensor. To overcome the insulating layer of skin, texture can be applied to the surface of the conductive tip that is applied to the sample site to aid in penetrating through insulating layers without puncturing the skin. Increased pressure may also be applied to the tip by the technician to aid in penetrating and compressing the insulating layer of skin. However, increased pressure leads to discomfort and pain during testing for many patients. Inaccuracies may also occur by variability between technicians that apply different forces while taking measurements using bioelectric measurement systems. As different forces are applied, contact between the conductive tip and the test point may vary, leading to inconsistent readings.

Vibration therapy has been incorporated in many health care modalities, where the rubbing or vibrating of an area can minimize pain and discomfort during procedures. For example, it is common for dentists to rub the gums of a patient where they are about to administer a shot to reduce the pain and discomfort caused by the shot. The use of vibration as a pain management technique falls under a theory called the “Gate Control Theory.”

The gate control theory of pain asserts that non-painful input closes nerve “gates” to painful input, which prevents pain sensation from traveling to the central nervous system. Under the Gate Control Theory, pain stimulation impulses are transmitted through small sensory fibers that enter the dorsal horn of the spinal cord. Then other cells, known as T-cells, transmit the impulses from the spinal cord up to the brain. Larger sensory fibers also carry stimulation to the spinal cord, where said stimulation is transmitted to the brain. In contrast to small sensory fibers, large sensory fibers carry harmless stimuli or mild irritations. The large sensory fibers can block or inhibit signals being sent to the brain through the small sensory fibers, effectively creating a “gate” that controls pain stimulation and signaling. These large sensory fibers are stimulated by harmless stimuli or mild irritations, including touching, rubbing, or light scratching of the skin. When large sensory fibers are more active due to stimulation, the “gate” is closed, and the pain stimulation from the small sensory fibers is not transmitted. In contrast, when small sensory are more active due to pain stimulation and signaling, without the activation of the large sensory fibers, the “gate” is open, and the pain stimulation can be sent to the brain where it is received, processed, and results in the pain sensation.

According to the theory, the gate can sometimes be overwhelmed by a large number of small, activated fibers. In other words, the greater the level of pain stimulation, the less adequate the “gate” of the large sensory fibers is in blocking the communication of this information.

In addition to reducing pain and discomfort during health care procedures, vibration feedback can be utilized to improve communication between a device and test subjects during use. The timing and frequency of different pulses can effectively and efficiently communicate information to the technician to provide immediate feedback to either a patient/test subject or the technician performing a procedure.

In light of the need for more accurate bioelectric measurement systems that can obtain consistent measurements while minimizing patient pain and discomfort, disclosed herein are systems, methods, and devices for incorporating vibration therapy and vibration feedback into a bioelectric test device or probe for measuring skin conductivity.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

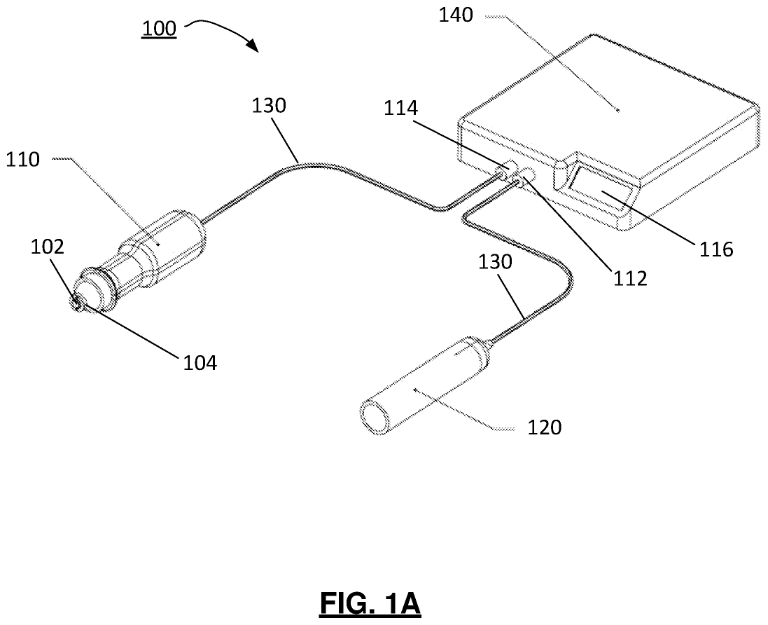

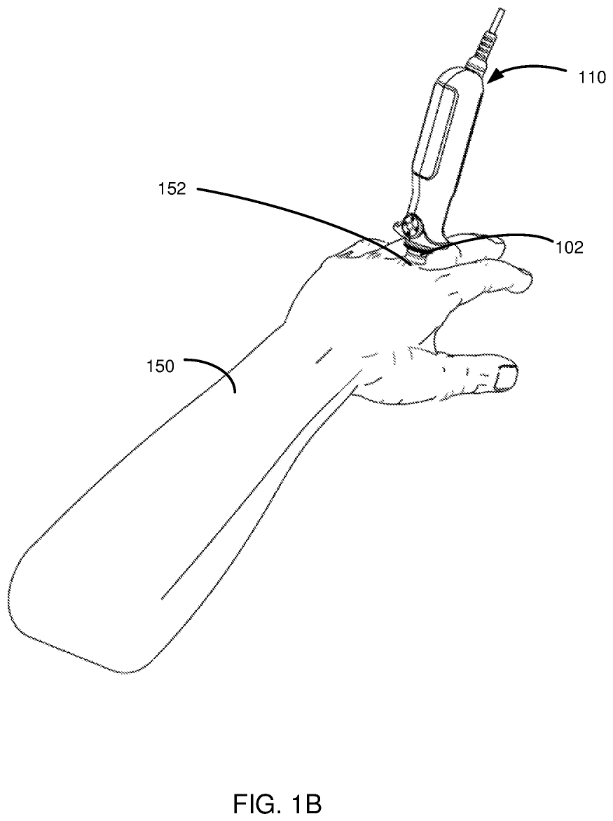

Now referring to the figures, FIG. 1A illustrates a perspective view of a bioelectric measurement system 100. As shown, bioelectric measurement system includes an electrodermal/bioelectric test probe 110, a grounding device 120 such as a hand mass, and a bioelectric measurement analyzing device 140 in electrical communication with bioelectric test probe 110 and grounding device 120.

Grounding device 120 may include a hand-held mass or hand mass to be held by a test subject undergoing measurements by bioelectric measurement system 100. Grounding device 120 may include a rod made of brass, or any other suitable material for grounding the test subject. Grounding device 120 may include a grounding surface disposed around an exterior of grounding device 120.

Grounding device 120 may be a small electrode similar to those used in conjunction with an electrocardiogram (EKG). Grounding device 120 may be any suitable size or shape and may be formed in an ergonomic size and shape that is easy for a test subject to hold in a palm of the test subject's hand. The grounding surface may be of a sufficiently large size to provide ample grounding to take sufficiently consistent and sufficiently accurate measurements from the test subject.

Bioelectric test probe 110 is configured to measure the resistance of skin, a meridian pathway in a body, acupressure points, or other materials or tissues of a test subject. The readings taken by bioelectric test probe 110 can be assessed to calculate the conductivity of the skin, the meridian pathway in the body, or other materials or tissues in the body. Bioelectric test probe 110 may include a testing end, which may include a probe hood 104 and a probe tip 102 disposed at a distal end, or in other words testing end, of bioelectric test probe 110 with respect to the electrical connection with bioelectric measurement analyzing device 140. Probe tip 102 may be placed against the skin of a test subject to enable bioelectric test probe 110 to measure the resistance of the skin or meridian pathway in the test subject. In an embodiment, bioelectric test probe 110 may take a measurement when probe tip 102 is pressed against tissue. Probe tip 102 may be constructed of any suitably electrically conductive material such as copper, silver, gold, aluminum, zinc, nickel, brass, iron, steel, or other material known to those skilled in the art.

In an embodiment, probe tip 102 is a single probe tip. In an alternative embodiment, probe tip 102 includes a plurality of individual probe tips. Probe tip 102 may be textured to help penetrate and help electricity flow through the insulation layer or cornified layer of the epithelial tissue without puncturing it. In another embodiment, grounding pads and/or contacts may be integrated with probe hood 104. Bioelectric test probe 110 may further include a contact sensor disposed on probe hood 104 to ensure contact between a test subject and bioelectric test probe 110.

The bioelectric measurement analyzing device 140 may include one or more processors configurable to execute instructions stored in non-transitory computer readable storage media. Bioelectric measurement system 100 may include memory stored locally therein and accessible by bioelectric measurement analyzing device 140. Bioelectric measurement analyzing device 140 is in electrical communication with bioelectric test probe 110, grounding device 120, and a display 116.

In the illustration shown in FIG. 1A, bioelectric measurement analyzing device 140 is in electrical communication with bioelectric test probe 110 by way of a sensor connection point 114 and is in electrical communication with grounding device 120 by way of grounding connection point 112. The electrical communication between bioelectric measurement analyzing device 140 and bioelectric test probe 110 and/or grounding device 120 can be facilitated by electrically conductive cables 130. In another embodiment, hand mass 120 may be attached to bioelectric measurement analyzing device 140, such that only one cable 130 is needed between bioelectric test probe 110 and bioelectric test device 140. Alternatively, the electrical communication may be made wirelessly through a wireless network such as a wireless personal area network (WPAN), a wireless local area network (WLAN), and so forth. Electrically conductive cables 130 may further be connected to a power source such that bioelectric test probe 110 and/or grounding device 120 are powered by way of an external power source.

According to one embodiment, conductance testing or measuring of meridian points may be done by having a test subject grip a conductive rod or hand mass (e.g., grounding device 120) in one hand while point readings are taken on the other hand and or the other side of the body using bioelectric test probe 110. Then the conductive rod (e.g., grounding device 120) may be placed in the other hand while the point readings are taken on the other side of the test subject's body with bioelectric test probe 110. Bioelectric test probe 110 may utilize probe tip 102 to contact the meridian points, and the readings may be taken and recorded while pressure is applied against the tissue at that point. As illustrated in FIG. 1B, the testing end of bioelectric test probe 110, including probe tip 102, may be pressed against body tissue or skin of test subject 150 at a test point 152. It is understood that test point 152 shown on test subject 150 in FIG. 1B is exemplary and not limiting. Test point 152 may be found anywhere else on test subject 150 or any other test subject which may be subjected to such testing, not just at the location shown in FIG. 1B.

A sequence in taking a meridian point reading from test subject 150 may be to first ground the test subject using grounding device 120 and then locate test point 152 and place probe tip 102 of bioelectric test probe 110, on that test point 152. The technician may then adjust the force and rate of force applied on probe tip 102 against test point 152 in a controlled manner in order to obtain sufficiently accurate and reliable measurements. This sequence can take about 5 seconds and sometimes up to 20 seconds.

Meridian points are located under the skin which adds to the difficulty in measuring resistance because skin can become dry and act as an insulator. To minimize this insulator effect, several methods may be utilized, either alone or in combination. One method may include spraying a mist of water or other conductive liquid on the hand that grips grounding device 120 to help increase the conductivity of the tissue that is gripping grounding device 120. Grounding device 120 is the ground or reference in the test circuit. Another method is to add moisture or water to the tissue where the reading is taking place with bioelectric test probe 110. Another method is to have a texture applied to the tip surface of probe tip 102 that helps penetrate the outer insulation layer of the skin or cornified layer of the epithelial tissue without puncturing it.

Another method to decrease the insulator effect of the skin is to adjust the pressure of probe tip 102 of bioelectric test probe 110 against the test subject's body tissue over the meridian point. Accordingly, taking a meridian point reading of a meridian point on a test subject's body may include locating test point 152, placing bioelectric test probe 110 on test point 152, and maintaining contact between probe tip 102 of bioelectric test probe 110 and the test subject's body tissue during testing/measuring while adjusting pressure of probe tip 102 of bioelectric test probe 110 against the test subject's body tissue. A practitioner/technician performing the measuring may adjust the force and rate of force of the tip of the bioelectric test probe 110 against the point in a controlled manner in order to obtain sufficiently accurate and reliable measurements.

However, as discussed previously, it takes much time, training, and practice for a practitioner/technician to get to the point where he/she can take sufficiently accurate and repeatable meridian point readings. There are many things that the technician has to be aware of including the conductivity of the skin being tested, controlling the proper rate of force, recognizing and acquiring the proper aspects of a curve and slope of the reading, locating the proper point locations, maintaining contact between the tip and the body tissue, and the angle of the tip. A typical technician can take six to twelve months, or more, of practice to become competent with electrical conductance diagnostic testing. Additionally, it may be difficult for an inexperienced technician to maintain sufficient continual contact between the electrodermal probe and the test subject's body tissue during bioelectric testing.

Difficulty in maintaining sufficient continual contact between the tip and the body tissue, frequent adjustments in pressure to maintain such contact, and difficulty in obtaining accurate and reliable readings may increase the time that a test subject is being tested and may also lead to discomfort and pain for the test subject depending on the length of testing and force between probe tip and the skin of the test subject.

This disclosure describes several systems, methods, devices, and computer program products to minimize the training time and improve accuracy and repeatability in meridian point readings by improving conductance between test probe and test subject and to relieve pain and discomfort for a test subject being tested.

For example, in at least one embodiment bioelectric test probe 110 may be an automated electrodermal probe that utilizes sensors, linear motors, and/or computerized controllers to properly control the force and rate of force probe tip 102 applies to test point 152 on test subject 150. In such a configuration, the force and rate of force probe tip 102 applies to test point 152 would be automated and controlled by a computerized system instead of the practitioner, thereby removing human error from testing and ensuring that proper contact and force is maintained between probe tip 102 of bioelectric test probe 110 and test subject 150.

As described in further detail below, an embodiment may incorporate vibration in the bioelectric test probe to improve electrical contact and conductance between the bioelectric test probe and meridian pathways in a test subject's skin by better penetrating the insulation layer of skin to reach a meridian point/pathway. Additionally, the vibration may alleviate pain and discomfort for the test subject during testing.

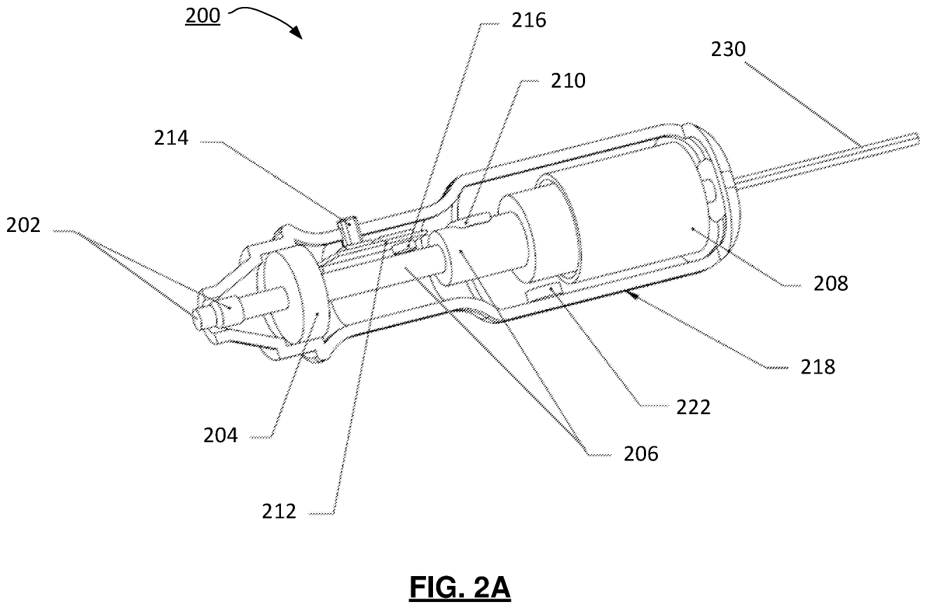

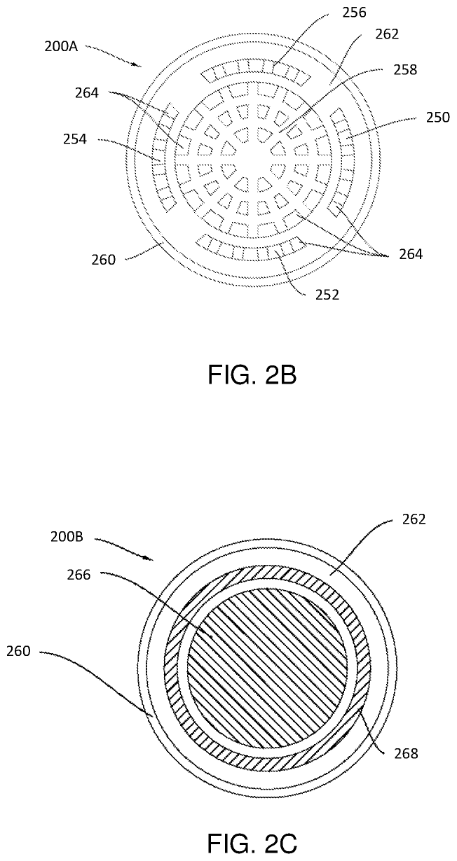

Referring now to FIG. 2A of the drawings, FIG. 2A illustrates a cutout perspective view of an embodiment of a bioelectric test probe 200. Bioelectric test probe 200 is configured with a probe tip that is a conductive tip 202 located at the distal end of bioelectric test probe 200 relative to a cable 230. Located adjacent to conductive tip 202 is a bearing 204. A shaft 206 runs through bearing 204, connecting conductive tip 202 to a motor 208. A shaft-mounted vibration device 210, such as a vibration motor, is located on shaft 206. A circuit board 212 is located parallel to shaft 206 and a switch 214 and circuit board mounted vibration device 216, such as a vibration motor, are located on circuit board 212. Bioelectric test probe 200 may further include a body mounted vibration device 222, such as a vibration motor. Any of the vibration devices may include a vibration motor, haptic motor, or other effective devices that produce a vibration or give tangible feedback.

Bioelectric test probe 200 is shown to include body-mounted vibration device 222, shaft-mounted vibration device 210, and circuit board mounted vibration device 216. Bioelectric test probe 200 may include any one or more of the listed vibration devices. Additionally, bioelectric test probe 200 may include further vibration devices disposed in various locations in bioelectric test probe 200. In an embodiment, the bioelectric test probe 200 may include a shaft mounted vibration device 210, and/or a vibration device 216 connected to the circuit board, or a vibration device 222 disposed of in the non-conductive probe body 218. In an embodiment, each of the shaft mounted vibration device 210 and the vibration device 216 and vibration device 222 operate independently under the direction of circuit board 212.

The various components of bioelectric test probe 200 are enclosed in a non-conductive probe body 218. Bioelectric test probe 200 is configured to measure the resistance of skin, acupressure points, a meridian pathway in a body, or other materials or tissues of a test subject. Conductive tip 202 can be applied to the skin surface to calculate the conductivity of the skin, acupressure points, meridian points, meridian pathways in the body, or other materials or tissues.

In an embodiment, conductive tip 202 is a single conductive tip. The conductive tip 202 may be textured to help penetrate the insulation layer or cornified layer of the tissue without puncturing it. In an alternative embodiment, the conductive tip 202 includes a plurality of individual tips. The one or more conductive tips 202 may be constructed of any suitably electrically conductive material such as copper, silver, gold, aluminum, zinc, nickel, brass, iron, steel, stainless steel, or other material known to those skilled in the art. In an embodiment, each of a plurality of conductive tips 202 can take an independent bioelectrical measurement. Conductive tips 202 may be further divided into one or more primary conductive tips located at the center and secondary conductive tips 202 that are positioned around one or more primary conductive tips located at the center of a tip of bioelectric test probe 200.

For example, FIGS. 2B and 2C show possible configurations of the bioelectric test probe 200A having multiple conductive tips. As shown in FIG. 2B, a plurality of ancillary tips including a first conductive tip 250, a second conductive tip 252, a third conductive tip 254, and a fourth conductive tip 256 are substantially equally spaced around a perimeter of the center primary conductive tip 258. While FIG. 2B shows that the first conductive tip 250, second conductive tip 252, third conductive tip 254, and fourth conductive tip 256 are arched in shape, generally any shape is acceptable. The center conductive tip 258 and the ancillary tips may extend out from the outer casing 260 between 0.1 to 10 mm, in some embodiments. Outer casing 260 may comprises support structure 262. Outer casing 260 and support structure 262 may be formed of a plastic or other nonconducting materials and are used to support or hold in position the conductive tips 250, 252, 254, 256, 258, and 268.

Center conductive tip 258 is located in the center of the sensor head. In the depicted embodiment, the primary conductive tip or center conductive tip 258 has a round shape and approximately three-fourths to half the diameter as the sensor head. As shown each of the conductive tips 250, 252, 254, 256, and 258 have multiple bristles 264. While FIG. 2B shows a uniform bristle 264 pattern, the bristle 264 pattern may be random. The bristles 264 may be manufactured in any manner including using methods such as welding, etching molding, electrical discharge machining (EDM), machining, stamping, rotary broach, or any other manner. The bristles 264 puncture the cornified layer of the epidermis to assist in obtaining the bioelectric conductance value(s). The bristles allow the measurement to be taken closer to the conductance point without causing damage to the skin, cause pain, or even bleeding. In other embodiments, the bristles 264 do not puncture the cornified layer and may optionally be used in combination with a material, such as water or gels, to enhance obtaining the bioelectric conductance value(s).

FIG. 2C illustrates a front view of an end of bioelectric test probe 200B having two conductive tips, according to one embodiment. Specifically, the sensor head includes a center conductive tip 266 and an ancillary conductive tip 268 encircling the center conductive tip 266. The arrangement of conductive tips may be used to determine whether the center conductive tip 266 is positioned over a conductance point/meridian point. For example, if the ancillary conductive tip 268 has a higher conductance reading (i.e., lower resistance or impedance) it can be determined that the conductance point is probably not located under the center conductive tip 266 and that the sensor head should be repositioned. As will be described further below, one of vibration devices 210, 216, and 222 may vibrate to provide an alert to a technician and/or patient that the probe tip needs to be repositioned.

When a tissue or meridian point is being tested, bioelectric test probe 200 is placed over the sample site and conductive tip 202 contacts the skin at the sample site creating a closed circuit. Subsequently, the automated bioelectric test probe 220 initiates the test cycle. Bioelectric test probe 200 then applies a force through motor 208 and shaft 206 to extend conductive tip 202 to contact the sample site and controls the applied force through the entire point test with no technician interference. The controlled force applied by motor 208 provides force throughout the procedure and eliminates error from a technician that may apply improper force while performing a measurement at a sample site.

As the skin conductivity measurement is taking place, readings are conveyed to circuit board 212 or through cable 230 to an EAV or similar device where the skin conductivity measurement may be assessed. It will be appreciated that significant force applied by the technician may be required to enable the conductive tip to compress and penetrate the cornified outer layer of skin at a test site to make an accurate skin conductivity measurement, resulting in discomfort and pain in many patients.

In light of this problem, in an embodiment, shaft-mounted vibration device 210 is affixed to shaft 206 to cause shaft 206 and conductive tip 202 to vibrate during certain stages of the test cycle. The vibration caused by shaft-mounted vibration device 210 may enable conductive tip 202 to penetrate the cornified layer of skin more readily and seat closer to the meridian point with less applied pressure, resulting in more reliable and more consistent readings as well as decreased discomfort and pain for the patient. In an embodiment shaft-mounted vibration device 210 may produce a single vibration pulse. In an alternative embodiment, shaft-mounted vibration device 210 may produce a plurality of vibration pulses. In an embodiment vibration device 210 may produce short vibration pulses when turned on, and in an alternative embodiment, vibration device 210 may produce long vibration pulses when turned on. The vibration pulses produced by shaft-mounted vibration device 210 could be made up of many combinations of these and other configurations (e.g., alternate between long and short pulses, produce one constant pulse, or use any number of long pulses followed by any number of short pulses in any conceivable alternating pattern).

The use of vibration as a pain management technique falls under a theory called the “Gate Control Theory.” The gate control theory of pain asserts that non-painful input closes nerve “gates” to painful input, which prevents pain sensation from traveling to the central nervous system. Under the Gate Control Theory, pain stimulation impulses are transmitted through small sensory fibers that enter the dorsal horn of the spinal cord. Then other cells, known as T-cells, transmit the impulses from the spinal cord up to the brain. Larger sensory fibers also carry stimulation to the spinal cord, where said stimulation is transmitted to the brain. In contrast to small sensory fibers, large sensory fibers carry harmless stimuli or mild irritations. The large sensory fibers can block or inhibit communication of stimulation signals being sent to the brain through the small sensory fibers, effectively creating a “gate” that controls pain stimulation and signaling. These large sensory fibers are stimulated by harmless stimuli or mild irritations, including touching, rubbing, or light scratching of the skin. When large sensory fibers are more active due to stimulation, the “gate” is closed, and the pain stimulation from the small sensory fibers is not transmitted. In contrast, when small sensory are more active due to pain stimulation and signaling, without the activation of the large sensory fibers, the “gate” is open, and the pain stimulation can be sent to the brain where it is received, processed, and results in the pain sensation.

There are multiple factors that contribute to whether pain stimulation is communicated to the brain. First, the amount of activity in the pain fibers or small sensory fibers may affect how pain is communicated to the brain. High activity in the small sensory fibers tends to open the gate, and the stronger the pain stimulation, the more active the small sensory fibers are, the more pain stimulation is communicated to the brain.

Second, the amount of activity in other peripheral fibers such as the large sensory fibers may affect the amount of pain stimulation that is communicated to the brain. The large sensory fibers are associated with harmless stimuli or mild irritation, such as touching, rubbing, or lightly scratching the skin. Activity in these kinds of stimulations will increase activity in the large sensory fibers and close the “gate” to painful stimulation communicated through small sensory fibers, thus, causing pain stimulation to not reach the brain as effectively.

Accordingly, applying a mild stimulus to the test location with vibration can help reduce the discomfort and pain that can come from painful, uncomfortable stimulation received from the force applied to the tip against meridian points during testing. Additionally, the vibration may allow the bioelectric test probe to penetrate the cornified layer of skin more effectively without puncturing the skin and to seat the conductive tip closer to the meridian point while reducing pain and discomfort for the patient during the test procedure.

In addition to pain management advantages obtained through vibration, vibration may be used as a form of alert and/or communication to inform and provide feedback to the technician and/or patient throughout the testing process to improve efficiency and accuracy. For example, such as in probe embodiments shown in FIGS. 2B and 2C, a vibration from one or more of vibration devices 210, 216, and 222 may provide feedback to the technician by signifying the accuracy of placement of bioelectric test probe 200 on the test subject. In order to obtain reliable and reasonably accurate conductance measurements from a meridian point of a test subject, a primary conductive tip should be placed accurately over a meridian point of the test subject.

Human tissue generally has a resistance of about 98,000 Ohms between the tissue and ground; meridian points have a general resistance of about 5,000 Ohms between the meridian point and ground. Accordingly, meridian points have a higher conductance and lower resistance than other human tissue. In order for a bioelectric test probe to be placed for accurate measurement of conductance/resistance of meridian points, center conductive tip 258 should be placed over a meridian point that is being measured. With respect to FIGS. 2B and 2C, if ancillary conductive tips 250, 252, 254, 256, and/or 268, which are electrically isolated from center conductive tip 258, measure a higher conductance reading then center conductive tip 258, then the ancillary conductive tips are closer to the meridian point then the center conductive tip 258. In such a case, bioelectric test probe 200 should be repositioned until center conductive tip 258 measures a higher conductance reading then ancillary conductive tips 250, 252, 254, 256, and/or 268 to ensure a more accurate resistance/conductance measurement of the meridian pathway is obtained.

If a situation arises where a higher conductance reading is at one or more ancillary conductive tips 250, 252, 254, 256, and/or 268 compared to center conductive tip 258, one or more of vibration devices 210, 216, and 222 may vibrate in a predetermined pattern to indicate that repositioning is required. Alternatively, 250, 252, 254, 256, and/or 268 one or more of vibration devices 210, 216, and 222 may vibrate in a predetermined pattern to indicate that positioning is correct and that center conductive tip 258 is positioned over a meridian pathway such that center conductive tip 258 has a higher conductance reading than ancillary conductive tips 250, 252, 254, 256, and/or 268.

In an embodiment, a vibration device 216 is mounted on the circuit board 212. Circuit board 212 may activate the circuit board mounted vibration device 216 following the pressing of switch 214 as a way of providing feedback to the technician holding the bioelectric test probe 200. In an alternative embodiment, the vibration device 216 may be disposed in the non-conductive probe body 218 to provide feedback to the technician through vibrations. In an embodiment vibration device 216 produces a single vibration pulse, and in an alternative embodiment, produces a plurality of vibration pulses at spaced intervals. In an embodiment vibration device 216 produces short vibration pulses, and in an alternative embodiment, vibration device 216 produces long vibration pulses. The vibration pulses produced by vibration device 216 could be made up of many combinations of these and other configurations to signal to the technician whether a measurement was properly taken or if it needs to be repeated. In an alternative embodiment, circuit board 212 may electronically communicate a vibration input through cable 230 to a grounding device 120 or hand mass held by the patient signifying that a measurement is about to be taken or that a measurement is complete. Likewise, vibration pulses may be produced by a body mounted vibration device or device.

For a technician, the vibration alerts/communication may communicate important information about the condition, proper positioning, operation, and operability of the device. This communication coupled with possible audible and visual communication can aid in the technician's confidence in achieving a higher level of quality and consistency in testing and measuring bioelectric information from a test subject.

Vibration alerts may also communicate important information to a patient being tested. For example, a small vibration to the patient through the probe or grounding device may communicate to the patient that testing has begun and focus the patient on sitting still and maintaining grip on the hand mass/grounding device. Another pulse may communicate to the patient that the test is done and allow the patient to relax or prepare for testing at the next test point.

The description is not limited to the above-described example for providing feedback to a technician using vibration devices 210, 216, and 222. Vibration devices 210, 216, and 222 may be utilized to provide feedback for any situation known in the art related to bioelectric test probes, devices, and systems. For example, a vibration may be provided signifying overheating of components of bioelectric measurement system 100. A vibration may be provided when a successful reading is obtained, or, alternatively, that a reading has failed. A vibration may be provided when electrical contact between a test subject and the probe is obtained, or when contact between the test subject and the probe is broken. A vibration may be provided signifying that a predetermined amount of time has elapsed. Any combination of the above situations may be implemented together with different vibration patterns being assigned to different alerts/situations. In short, vibration may be used as tactile feedback for any operation, operability, or malfunction of a bioelectric measurement system and its individual components.

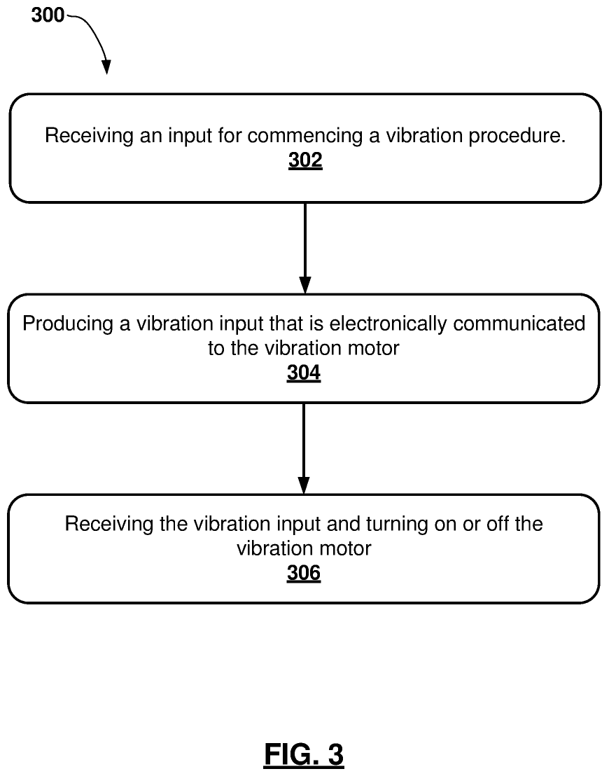

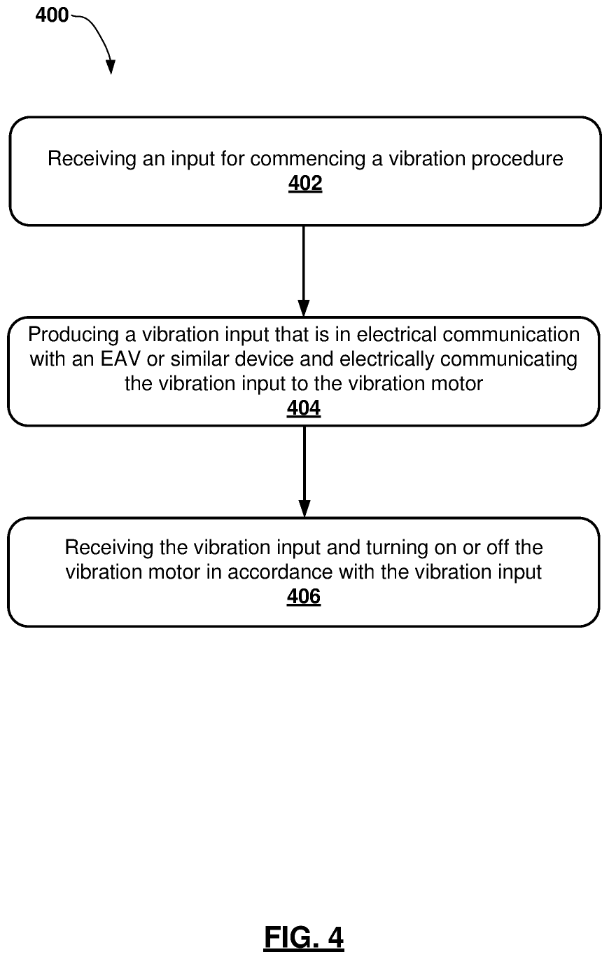

FIG. 3 is a schematic flow chart diagram of a method 300 for coupling vibration therapy with output force in a bioelectric test probe to penetrate the cornified layer of skin more effectively without puncturing the skin and seat the conductive tip closer to the meridian point while reducing pain and discomfort for the patient during the test procedure. The method 300 may be performed by any suitable computing device, such as one or more processors in circuit board 212 in electrical communication with a motor 208 and one or more vibration devices 210, 216, 222 in bioelectric test probe 200.

The method 300 begins with a bioelectric test probe contacting skin of a test subject who is in contact with a grounding device of a bioelectric testing system. With the test subject in contact with the test probe, an input is received at step 302 for commencing a vibration procedure. This input is communicated to a circuit board of the bioelectric testing system. Upon receiving the input for commencing the vibration procedure, one or more processors on the circuit board produces a vibration routine as vibration input that is electronically communicated from the circuit board to the vibration device (e.g., one or more vibration devices 210, 216, 222 in bioelectric test probe 200) at step 304. The method 300 continues as the vibration device receives the vibration input and the vibration device turns on or off at step 306 to aid in penetrating the outer cornified layer of skin during bioelectric testing, while reducing discomfort and pain in the patient. The bioelectric test probe may obtain bioelectric readings and measurements from the test subject during the vibration procedure while the probe is in contact with the test subject. The method repeats and continues throughout bioelectric testing and measurement of a test subject using the bioelectric test probe.

It will be appreciated that vibration routines may vary depending on the vibration procedure corresponding to the input and the purpose of the vibration routine needed. For example, the vibration procedure may be a routine set to provide a constant pulse during testing to alleviate pain and discomfort of the test subject. Alternatively, the vibration procedure may provide a certain pulse pattern (e.g., short pulses, long pulses, constant pulses, higher intensity pulses, lower intensity pulses, or any combination thereof) that is effective in alleviating pain in the test subject. The vibration procedure may also provide communication to the technician and/or patient by providing a vibration at the start of bioelectric testing, providing a vibration at the end of testing, providing a vibration during the length of testing that begins at the start of testing and ends at the end of testing.

FIG. 4 is a schematic flow chart diagram of a method 400 for incorporating vibration feedback in a bioelectric test probe to provide enhanced communication to the patient during the test procedure. The method 400 may be performed by any suitable computing device, such as one or more processors in circuit board 212 in electrical communication with a shaft mounted vibration device 210, and or a circuit board mounted vibration device 216 and/or a body mounted vibration device 222 in bioelectric test probe 200.

The method 400 may begin with a bioelectric test probe contacting skin of a test subject who is in contact with a grounding device of a bioelectric testing system. With the test subject in contact with the test probe, an input is received at step 402 for commencing a vibration procedure. The input is electronically communicated to the circuit board of a bioelectric testing system or test probe. Upon receiving the input for commencing the vibration procedure, one or more processors on the circuit board produces a vibration sequence as vibration input that is electronically communicated to an EAV or similar device and electronically communicating the vibration input to the vibration motor at step 404. Once the vibration input is received the vibration motor is turned on or off in accordance with the vibration input at step 406. The vibration may signal to the patient that a measurement is about to be taken, has begun being taken, is in the process of being taken, or has been completed. In one embodiment the vibration sequence is electronically communicated through a cable. In an alternative embodiment, the vibration sequence is electronic communication performed wirelessly. The bioelectric test probe may obtain bioelectric readings and measurements from the test subject during the vibration procedure while the probe is in contact with the test subject. The method repeats and continues throughout use of the bioelectric test probe.

It will be appreciated that vibration routines may vary depending on the vibration procedure corresponding to the input and the purpose of the vibration routine needed. For example, the vibration procedure may provide communication to the technician and/or patient by providing a vibration at the start of bioelectric testing, providing a vibration at the end of testing, providing a vibration during the length of testing that begins at the start of testing and ends at the end of testing. Additionally or alternatively, the vibration procedure may provide a pulse of a first intensity to signal commencement of taking a bioelectric measurement, may provide a pulse of a second intensity during testing, and may provide a pulse of a third intensity when testing ends to keep a test subject aware of the stage of testing. The third and first intensities may be the same or different from each other. This procedure provides an advantage of keeping the test subject aware that testing is being performed so that they may focus on remaining still and in contact with the probe and grounding device during testing and will also allow the test subject to relax between readings and/or when testing is completed.

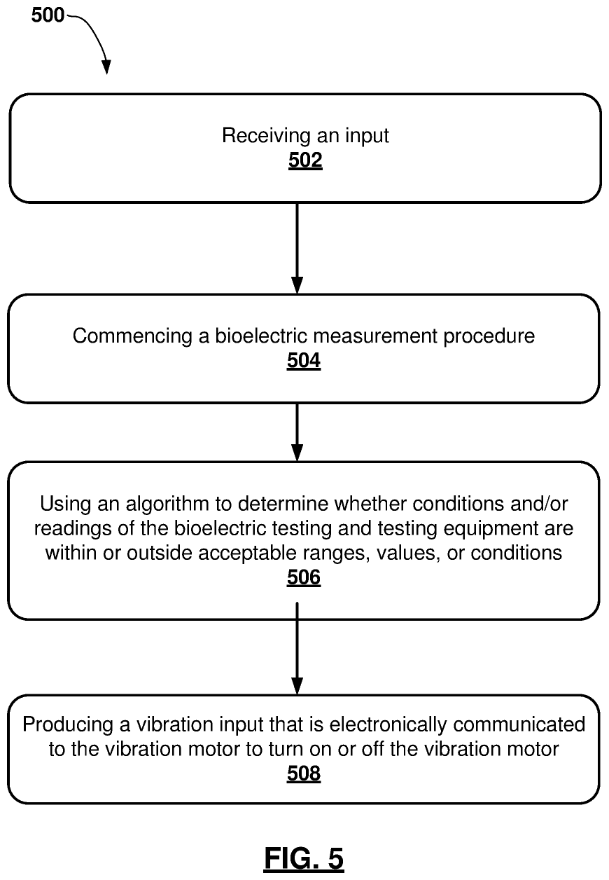

FIG. 5 is a schematic flow chart diagram of a method 500 for incorporating vibration feedback in a bioelectric test probe to provide enhanced communication to the technician performing the test procedure. The method 500 may be performed by any suitable computing device, such as one or more processors in circuit board 212 in electrical communication with a motor 208 and circuit board mounted vibration device 216 and or a shaft mounted vibration device 210 and or a body mounted vibration device 222 in bioelectric test probe 200.

The method 500 may begin with a bioelectric test probe contacting skin of a test subject who is in contact with a grounding device of a bioelectric testing system. The method 500 begins and an input for commencing a bioelectric measurement procedure is received at step 502 that is communicated to the circuit board of a bioelectric testing system or test probe. Upon receiving the input for commencing the bioelectric measurement procedure, one or more processors on the circuit board commences the procedure 504. The method 500 continues as the circuit board uses an algorithm at step 506 to determine whether conditions/readings of the bioelectric testing and testing equipment are within or outside acceptable ranges, values, or conditions. The circuit board then produces a vibration input at step 508 that is transmitted to a vibration device, such as one or more of vibration device 216 mounted to the circuit board, vibration device 222 disposed in the non-conductive housing, and/or the vibration device 210 mounted to the shaft, to turn the motor on or off to communicate with the technician whether conditions and/or readings of the test are within appropriate ranges or values. The method repeats and continues throughout use of the bioelectric test probe.

It will be appreciated that the algorithms used in step 506 may vary depending on the conditions being monitored during testing. For example, the vibration procedure may provide communication to the technician by providing a vibration at the start of bioelectric testing, providing a vibration at the end of testing, providing a vibration during the length of testing that begins at the start of testing and ends at the end of testing. Additionally or alternatively, the vibration procedure may provide a pulse of a first intensity to signal commencement of taking a bioelectric measurement, may provide a pulse of a second intensity during testing, and may provide a pulse of a third intensity when testing ends to keep a test subject aware of the stage of testing. The third and first intensities may be the same or different from each other. This procedure provides an advantage of keeping the technician aware that testing is being done so that they may focus on keeping a probe still during testing.

Alternatively or additionally, the algorithm may monitor communicate important information about the condition, proper positioning, operation, and operability of the device. For example, a vibration may be provided signifying overheating of components of the bioelectric measurement system or test probe. A vibration may be provided when a successful reading is obtained, or, alternatively, that a reading has failed. A vibration may be provided when a reading is inside an expected range. A vibration may be provided when a reading is outside an expected range. A vibration may be provided when electrical contact between a test subject and the probe is obtained, or when contact between the test subject and the probe is broken. A vibration may be provided when one or more components in the system or probe is detected to be malfunctioning. A vibration may be provided signifying that a predetermined amount of time has elapsed.

Additionally, as described elsewhere in this description, one or more vibrations may be provided signifying proper and/or improper positioning of conductive tips of the test probe based on conductance/resistance readings from a primary conductive tip in conjunction with conductance/resistance readings from one or more ancillary conductive tips (e.g., as shown in FIGS. 2B and 2C). For example, if the conductive tip is properly positioned over a meridian point of a test subject, a vibration indicating proper positioning may be provided. Alternatively, a vibration may be provided signifying improper positioning of the primary conductive tip. Any combination of the above situations may be implemented together with different vibration patterns being assigned to different alerts/situations. In short, vibration may be used as tactile feedback for any operation, operability, or malfunction of a bioelectric measurement system and its individual components.

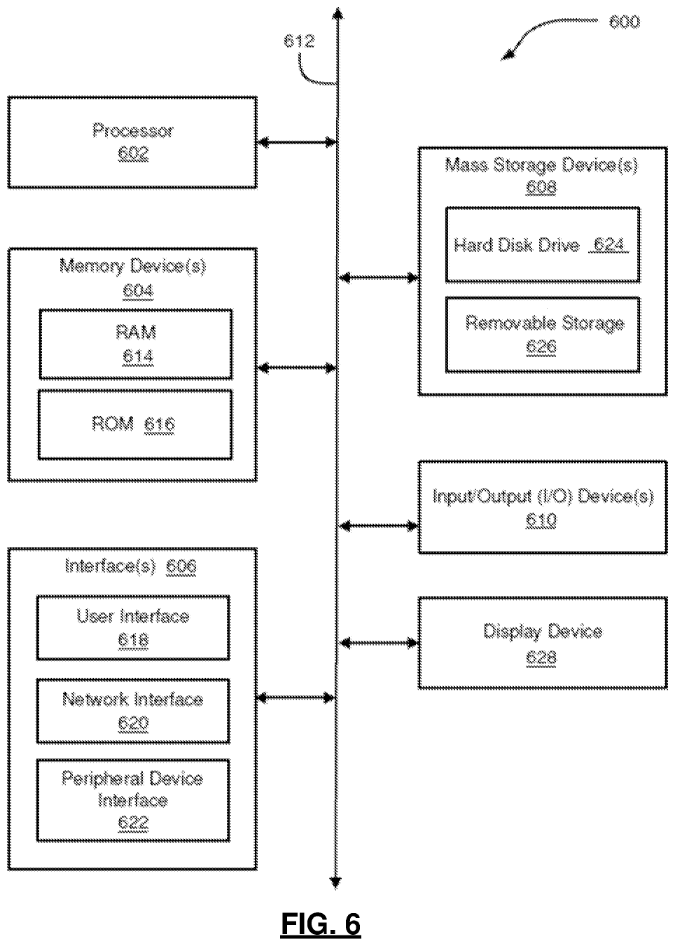

FIG. 6 is a block diagram illustrating an example computing device 600. Computing device 600 may be used to perform various procedures, such as those discussed herein. Computing device 600 can function as a server, a client, or any other computing entity such as the printed circuit board 212 in communication with the bioelectric test probe 200. Computing device can perform various monitoring functions as discussed herein, and can execute one or more application programs, such as the application programs described herein. Computing device 600 can be any of a wide variety of computing devices, such as probe printed circuit board 212, a desktop computer, a notebook computer, a server computer, a handheld computer, tablet computer and the like.

Computing device 600 may include one or more processor(s) 602, one or more memory device(s) 604, one or more interface(s) 606, one or more mass storage device(s) 608, one or more Input/Output (I/O) device(s) 610, and a display device 628 all of which are coupled to a bus 612. Processor(s) 602 include one or more processors or controllers that execute instructions stored in memory device(s) 604 and/or mass storage device(s) 608. Processor(s) 602 may also include various types of computer-readable media, such as cache memory.

Memory device(s) 604 include various computer-readable media, such as volatile memory (e.g., random access memory (RAM) 614) and/or nonvolatile memory (e.g., read-only memory (ROM) 616). Memory device(s) 604 may also include rewritable ROM, such as Flash memory.

Mass storage device(s) 608 include various computer readable media, such as magnetic tapes, magnetic disks, optical disks, solid-state memory (e.g., Flash memory), and so forth. As shown in FIG. 6, a particular mass storage device is a hard disk drive 624. Various drives may also be included in mass storage device(s) 608 to enable measurement from and/or writing to the various computer readable media. Mass storage device(s) 608 include removable media 626 and/or non-removable media.

I/O device(s) 610 include various devices that allow data and/or other information to be input to or retrieved from computing device 600. Example I/O device(s) 610 include cursor control devices, keyboards, keypads, microphones, monitors or other display devices, speakers, printers, network interface cards, modems, lenses, CCDs or other image capture devices, and the like.

Display device 628 includes any type of device capable of displaying information to one or more users of computing device 600. Examples of display device 628 include a monitor, display terminal, video projection device, and the like.

Interface(s) 606 include various interfaces that allow computing device 600 to interact with other systems, devices, or computing environments. Example interface(s) 606 may include any number of different network interfaces 620, such as interfaces to local area networks (LANs), wide area networks (WANs), wireless networks, and the Internet. Other interface(s) include user interface 618 and peripheral device interface 622. The interface(s) 606 may also include one or more user interface elements 618. The interface(s) 606 may also include one or more peripheral interfaces such as interfaces for printers, pointing devices (mice, track pad, or any suitable user interface now known to those of ordinary skill in the field, or later discovered), keyboards, and the like.

Bus 612 allows processor(s) 602, memory device(s) 604, interface(s) 606, mass storage device(s) 608, and I/O device(s) 610 to communicate with one another, as well as other devices or components coupled to bus 612. Bus 612 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 600 and are executed by processor(s) 602. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

Implementations of the disclosure may comprise or utilize a special purpose or general-purpose computer including computer hardware, such as, for example, one or more processors and system memory, as discussed in greater detail below. Implementations within the scope of the disclosure also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media devices or vice versa. For example, computer-executable instructions or data structures received over a network or data link can be buffered in RAM 614 within a network interface module 620 (e.g., a “NIC”), and then eventually transferred to computer system RAM 614 and/or to less volatile computer storage media (devices) at a computer system. RAM 614 can also include solid state drives (SSDs or PCIx based real time memory tiered storage, such as FusionIO). Thus, it should be understood that computer storage media devices can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the disclosure can also be used in cloud computing environments. In this description and the following claims, “cloud computing” is defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, or any suitable characteristic now known to those of ordinary skill in the field, or later discovered), service models (e.g., Software as a Service (SaaS), Platform as a Service (PaaS), Infrastructure as a Service (IaaS)), and deployment models (e.g., private cloud, community cloud, public cloud, hybrid cloud, or any suitable service type model now known to those of ordinary skill in the field, or later discovered). Databases and servers described with respect to the disclosure can be included in a cloud model.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the following description and Claims to refer to particular system components. As one skilled in the art will appreciate, components may be referred to by different names. This document does not intend to distinguish between components that differ in name, but not function.

### EXAMPLES

The following examples pertain to further embodiments.

Example 1 is a bioelectric test probe, wherein the bioelectric test probe comprises a non-conductive housing, a conductive tip connected to a shaft and a motor, a circuit board that communicates with the motor and a vibration device, wherein the motor applies controlled force to the conductive tip to determine skin conductivity, and wherein the vibration device produces vibrations within the bioelectric test probe.

Example 2 is a device as in Example 1, wherein the instructions sent to the vibration device operate to turn the vibration device on or off depending upon an input initiated from a test tip contacting a subject tissue creating a closed circuit or pressing of a switch.

Example 3 is a device as in any of Examples 1-2, wherein the vibration device is connected to the shaft to vibrate the shaft and conductive tip.

Example 4 is a device as in any of Examples 1-3, wherein the vibration device is attached to the circuit board.

Example 5 is a device as in any of Examples 1-4, wherein the vibration device is disposed of in the non-conductive housing to provide optimal vibration in the non-conductive housing of the bioelectric test probe.

Example 6 is a device as in any of Examples 1-5, wherein the bioelectric test probe includes a plurality of vibration devices including vibration devices connected to the shaft, on the circuit board, and connected to the non-conductive housing.

Example 7 is a device as in any of Examples 1-6, wherein the conductive tip is rigidly connected to the shaft and motor and wherein the motor includes a controller to regulate output force.

Example 8 is a device as in any of Examples 1-7, wherein the bioelectric test probe comprises a plurality of conductive tips for sensing conductivity of tissue, wherein each of the plurality of conductive tips is independent and makes an independent measurement of conductivity of a tissue.

Example 9 is a device as in any of Examples 1-8, wherein the non-conductive body incorporates an isolating hood that surrounds the conductive tip and isolates the technician from the probe.

Example 10 is a device as in any of Examples 1-9, wherein the test tip or switch produces an input that is electronically conveyed to a processor on the printed circuit board.

Example 11 is a device as in any of Examples 1-10, wherein the processor produces a vibration input based off of the input that is electronically conveyed to the vibration device to turn the vibration device on or off.

Example 12 is a device as in any of Examples 1-11, wherein a processor compares the parameters of a bio-conductance measurement to parameters of an acceptable bio-conductance measurement and produces a vibration input that turns on the vibration device to signal to the technician the measurements condition or if it was acceptable.

Example 13 is a device as in any of Examples 1-12, wherein the processor produces a vibration input that is electronically communicated through the cable to a connected EAV or similar device to signal to the patient that a measurement is starting or completed.

Example 14 is a device as in any of Examples 1-13, wherein the processor produces a vibration input that is wirelessly transmitted to an EAV or similar device to signal to the patient that a measurement is starting or completed.

Example 15 is a method. The method includes producing an input based on a switch being pressed by the technician using the bioelectric test probe.

Example 16 is a method as in Example 15, wherein the method includes producing an input based on the test tip contacting a test subject's tissue or a switch being pressed by the technician using the bioelectric test probe and electronically conveying the input to the circuit board.

Example 17 is a method as in Examples 15-16. The method includes receiving an input from the test tip or switch and producing a vibration input that is electronically communicated to the shaft mounted vibration device to turn the motor on or off using a processor.

Example 18 is a method as in Examples 15-17. The method includes receiving an input from the test tip or switch and producing a vibration input that is electronically communicated to an EAV or similar device.

Example 19 is a method as in Examples 15-18. The method includes receiving an input from the test tip or switch and producing a vibration input that is electronically communicated through a cable to an EAV or similar device.

Example 20 is a method as in Examples 15-19. The method includes receiving an input from the test tip or switch and producing a vibration input that is electronically communicated wirelessly to an EAV or similar device.

Example 21 is a method as in Examples 15-20. The method includes receiving an input from the test tip or switch and comparing the skin conductivity measurement to an acceptable range of conductivity measurements using a processor.

Example 22 is a method as in Examples 15-21, wherein after receiving the input and comparing the skin conductivity measurement to an acceptable range of conductivity measurements, the processor produces a vibration input that is electronically communicated to a vibration device connected to the circuit board or a vibration device disposed of in the non-conductive housing to turn the motor on or off.

Example 23 is non-transitory computer readable storage media storing instructions to be executed by one or more processors, the instructions comprising: receiving an input from a test tip or a switch; and producing a vibration input to be electronically communicated to the shaft mounted vibration device to turn on the vibration device to vibrate the shaft, conductive tip, and the whole probe.

Example 24 is non-transitory computer readable storage media as in Example 23, the instructions comprising: receiving an input from the test tip or switch; using an algorithm to compare parameters of a bio-conductance measurement to acceptable parameters of a bio-conductance measurement; and producing a vibration input to be electronically communicated to the vibration device connected to the circuit board for communication with the technician using the bioelectric test probe.

Example 25 is non-transitory computer readable storage media as in any of Examples 23-24, the instructions comprising: receiving an input; using an algorithm to compare parameters of a bio-conductance measurement to acceptable parameters of a bio-conductance measurement; and producing a vibration input to be electronically communicated to the vibration device disposed within the non-conductive housing to provide optimal vibration for communication with the technician using the bioelectric test probe.

Example 26 is non-transitory computer readable storage media as any of Examples 23-25, the instructions comprising: receiving an input; using an algorithm to compare parameters of a bio-conductance measurement to acceptable parameters of a bio-conductance measurement; and producing a vibration input to be electronically communicated to a connected EAV or similar device for communication with the patient during the test procedure.

Example 27 is non-transitory computer readable storage media as any of Examples 23-26, the instructions comprising: receiving an input; using an algorithm to compare parameters of a bio-conductance measurement to acceptable parameters of a bio-conductance measurement; and producing a vibration input to be electronically communicated through a cable to a connected EAV or similar device for communication with the patient during the test procedure.

Example 28 is non-transitory computer readable storage media as any of Examples 23-27, the instructions comprising: receiving an input; using an algorithm to compare parameters of a bio-conductance measurement to acceptable parameters of a bio-conductance measurement; and producing a vibration input to be wirelessly electronically communicated to a connected EAV or similar device for communication with the patient during the test procedure.

Example 29 is a bioelectric testing device. The bioelectric testing device may include a conductive tip that is disposed at a distal end of the bioelectric testing device and one or more vibration motors for vibrating the bioelectric testing device during bioelectric testing of a test subject.

Example 30 is a bioelectric testing device as in Example 29, wherein the bioelectric testing device may further include a motor that applies a motor output force to the conductive tip during bioelectric testing to maintain proper force between a test subject and the conductive tip during the bioelectric testing.

Example 31 is bioelectric testing device as in any of Examples 29-30, wherein the bioelectric testing device may further include a controller for automating the motor output force applied to the conductive tip by the motor during bioelectric testing to maintain proper force between the test subject and the conductive tip during the bioelectric testing.

Example 32 is bioelectric testing device as in any of Examples 29-31, wherein the one or more vibration motors may include one or more of a first vibration motor mounted on a shaft of the conductive tip for vibrating the conductive tip during bioelectric testing; a second vibration motor mounted on a housing of the bioelectric testing device that houses components of the bioelectric testing device; and a third vibration motor mounted on a circuit board within the housing of the bioelectric testing device.

Example 33 is bioelectric testing device as in any of Examples 29-32, wherein the bioelectric testing device may further include a non-conductive housing comprising a non-conductive body; and an isolating hood surrounding the conductive tip to isolate a technician from other elements of the bioelectric testing device.

Example 34 is bioelectric testing device as in any of Examples 29-33, wherein the one or more vibration motors vibrate to provide alerts about operation or operability of the bioelectric testing device to a technician or test subject.

Example 35 is bioelectric testing device as in any of Examples 29-34, wherein the conductive tip of the bioelectric testing device is a primary conductive tip. The bioelectric testing device further comprises one or more secondary conductive tips. The conductive tips may include the primary conductive tip and the one or more secondary conductive tips. The conductive tips may be electrically isolated from each other such that each conductive tip obtains independent bioelectric measurements; and wherein the one or more vibration motors vibrate to provide an alert indicating which conductive tip of the primary conductive tip and the one or more secondary conductive tips obtain a higher bioelectric reading.

Example 36 is a bioelectric testing system for taking bioelectric measurements of a test subject, the bioelectric testing system may include a grounding device for contacting a test subject, a bioelectric test probe, and a controller for automating operation of the bioelectric testing system. The bioelectric test probe may include a conductive tip that is disposed at a distal end of the bioelectric test probe and one or more vibration motors for vibrating the bioelectric test probe during bioelectric testing of a test subject.

Example 37 is bioelectric testing system as in Example 36, wherein the bioelectric test probe may further include a motor that applies a motor output force to the conductive tip during bioelectric testing to maintain proper force between a test subject and the conductive tip during the bioelectric testing.

Example 38 is bioelectric testing system as in any of Examples 36-37, wherein the bioelectric testing system may further include a controller for automating the motor output force applied to the conductive tip by the motor during bioelectric testing to maintain proper force between the test subject and the conductive tip during the bioelectric testing.

Example 39 is bioelectric testing system as in any of Examples 36-38, wherein the one or more vibration motors may include one or more of a first vibration motor mounted on a shaft of the conductive tip for vibrating the conductive tip during bioelectric testing, a second vibration motor mounted on a housing of the bioelectric test probe that houses components of the bioelectric test probe, and a third vibration motor mounted on a circuit board within the housing of the bioelectric test probe.

Example 40 is bioelectric testing system as in any of Examples 36-39, wherein the bioelectric test probe may further include a non-conductive housing comprising a non-conductive body and an isolating hood surrounding the conductive tip to isolate a technician from other elements of the bioelectric test probe.

Example 41 is bioelectric testing system as in any of Examples 36-40, wherein the one or more vibration motors vibrate to provide alerts about operation or operability of the bioelectric test probe to a technician or test subject.

Example 42 is bioelectric testing system as in any of Examples 36-41, wherein the conductive tip of the bioelectric testing device is a primary conductive tip. The bioelectric testing device may further include one or more secondary conductive tips. The conductive tips, including the primary conductive tip and the one or more secondary conductive tips, may be electrically isolated from each other such that each conductive tip obtains independent bioelectric measurements. The one or more vibration motors may vibrate to provide an alert indicating which conductive tip of the primary conductive tip and the one or more secondary conductive tips obtain a higher bioelectric reading.

Example 43 is a method for taking bioelectric measurements of a test subject with a bioelectric testing device that may include a conductive tip that is disposed at a distal end of the bioelectric testing device, and one or more vibration motors for vibrating the bioelectric testing device. The method may include contacting a test subject with the conductive tip of the bioelectric testing device, vibrating, with the one or more vibration motors, the bioelectric testing device, and taking one or more bioelectric measurements of the test subject with the bioelectric testing device.

Example 44 is a method as in Example 43, further including applying, with a motor, a motor output force to the conductive tip during bioelectric testing to maintain proper force between a test subject and the conductive tip during the bioelectric testing.

Example 45 is a method as in any of Examples 43-44, further including providing an alert about the operation or operability of the bioelectric testing system to a technician or test subject by vibrating the one or more vibration motors.

Example 46 is a method as in any of Examples 43-45, wherein the conductive tip of the bioelectric testing device is a primary conductive tip, and the bioelectric testing device may further include one or more secondary conductive tips. The conductive tips, including the primary conductive tip and the one or more secondary conductive tips, may be electrically isolated from each other such that each conductive tip obtains independent bioelectric measurements. The method may further include providing an alert indicating which conductive tip of the primary conductive tip and the one or more secondary conductive tips obtain a higher bioelectric reading by vibrating the one or more vibration motors.

Example 47 is a device, system, or method as in any of Examples 1-46, wherein the one or more vibration motors vibrate the conductive tip.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A bioelectric testing device comprising:
a conductive tip that is disposed at a distal end of the bioelectric testing device; and
one or more vibration motors for vibrating the bioelectric testing device during bioelectric testing of a test subject.

2. The bioelectric testing device according to claim 1, further comprising:
a motor that applies a motor output force to the conductive tip during bioelectric testing to maintain proper force between a test subject and the conductive tip during the bioelectric testing.

3. The bioelectric testing device according to claim 2, further comprising:
a controller for automating the motor output force applied to the conductive tip by the motor during bioelectric testing to maintain proper force between the test subject and the conductive tip during the bioelectric testing.

4. The bioelectric testing device according to claim 1,
wherein the one or more vibration motors comprise one or more of:
a first vibration motor mounted on a shaft of the conductive tip for vibrating the conductive tip during bioelectric testing;
a second vibration motor mounted on a housing of the bioelectric testing device that houses components of the bioelectric testing device; and
a third vibration motor mounted on a circuit board within the housing of the bioelectric testing device.

5. The bioelectric testing device according to claim 1, further comprising:
a non-conductive housing comprising:
a non-conductive body; and
an isolating hood surrounding the conductive tip to isolate a technician from other elements of the bioelectric testing device.

6. The bioelectric testing device according to claim 1, wherein the one or more vibration motors vibrate to provide alerts about operation or operability of the bioelectric testing device to a technician or test subject.

7. The bioelectric testing device according to claim 6, wherein the conductive tip of the bioelectric testing device is a primary conductive tip;
wherein the bioelectric testing device further comprises one or more secondary conductive tips;
wherein the conductive tips, including the primary conductive tip and the one or more secondary conductive tips, are electrically isolated from each other such that each conductive tip obtains independent bioelectric measurements; and
wherein the one or more vibration motors vibrate to provide an alert indicating which conductive tip of the primary conductive tip and the one or more secondary conductive tips obtain a higher bioelectric reading.

8. The bioelectric testing device according to claim 1, wherein the one or more vibration motors vibrate the conductive tip.

9. A bioelectric testing system for taking bioelectric measurements of a test subject, the bioelectric testing system comprising:
a grounding device for contacting a test subject;
a controller for automating operation of the bioelectric testing system;
a bioelectric test probe comprising:
a conductive tip that is disposed at a distal end of the bioelectric test probe; and
one or more vibration motors for vibrating the bioelectric test probe during bioelectric testing of a test subject.

10. The bioelectric testing system according to claim 9, the bioelectric test probe further comprising:
a motor that applies a motor output force to the conductive tip during bioelectric testing to maintain proper force between a test subject and the conductive tip during the bioelectric testing.

11. The bioelectric testing system according to claim 10, further comprising:
a controller for automating the motor output force applied to the conductive tip by the motor during bioelectric testing to maintain proper force between the test subject and the conductive tip during the bioelectric testing.

12. The bioelectric testing system according to claim 9,
wherein the one or more vibration motors comprise one or more of:
a first vibration motor mounted on a shaft of the conductive tip for vibrating the conductive tip during bioelectric testing;
a second vibration motor mounted on a housing of the bioelectric test probe that houses components of the bioelectric test probe; and
a third vibration motor mounted on a circuit board within the housing of the bioelectric test probe.

13. The bioelectric testing system according to claim 9, the bioelectric test probe further comprising:
a non-conductive housing comprising:
a non-conductive body; and
an isolating hood surrounding the conductive tip to isolate a technician from other elements of the bioelectric test probe.

14. The bioelectric testing system according to claim 9, wherein the one or more vibration motors vibrate to provide alerts about operation or operability of the bioelectric test probe to a technician or test subject.

15. The bioelectric testing system according to claim 14, wherein the conductive tip of the bioelectric testing device is a primary conductive tip;
wherein the bioelectric testing device further comprises one or more secondary conductive tips;
wherein the conductive tips including the primary conductive tip and the one or more secondary conductive tips are electrically isolated from each other such that each conductive tip obtains independent bioelectric measurements; and
wherein the one or more vibration motors vibrate to provide an alert indicating which conductive tip of the primary conductive tip and the one or more secondary conductive tips obtain a higher bioelectric reading.

16. The bioelectric testing system according to claim 9, wherein the one or more vibration motors vibrate the conductive tip.

17. A method for taking bioelectric measurements of a test subject with a bioelectric testing device comprising a conductive tip that is disposed at a distal end of the bioelectric testing device, and one or more vibration motors for vibrating the bioelectric testing device, wherein the method comprises:
contacting a test subject with the conductive tip of the bioelectric testing device;
vibrating, with the one or more vibration motors, the bioelectric testing device; and
taking one or more bioelectric measurements of the test subject with the bioelectric testing device.

18. The method of claim 17, further comprising:
applying, with a motor, a motor output force to the conductive tip during bioelectric testing to maintain proper force between a test subject and the conductive tip during the bioelectric testing.

19. The method of claim 17, further comprising:
providing an alert about the operation or operability of the bioelectric testing system to a technician or test subject by vibrating the one or more vibration motors.

20. The method of claim 19, wherein the conductive tip of the bioelectric testing device is a primary conductive tip;
wherein the bioelectric testing device further comprises one or more secondary conductive tips;
wherein the conductive tips, including the primary conductive tip and the one or more secondary conductive tips, are electrically isolated from each other such that each conductive tip obtains independent bioelectric measurements; and
wherein the method further comprises:
providing an alert indicating which conductive tip of the primary conductive tip and the one or more secondary conductive tips obtain a higher bioelectric reading by vibrating the one or more vibration motors.
